(19) United States
(12) Patent Application Publication
(10) Pub. No.: US 2012/0034641 A1
(43) Pub. Date: Feb. 9, 2012

(54) BIOLOGICAL INFORMATION ACQUISITION METHOD

(75) Inventor: Kazuhiro Nakagawa, Saitama (JP)

(73) Assignee: SONY CORPORATION, Tokyo (JP)

(21) Appl. No.: 13/084,724

(22) Filed: Apr. 12, 2011

(30) Foreign Application Priority Data
Aug. 4, 2010 (JP) P2010-175710

(51) Int. Cl. C12Q 1/02; G01N 33/48

(52) U.S. Cl. 435/29; 73/61.43

(57) ABSTRACT

A biological information acquisition method includes: measuring an amount of nicotinamide metabolite in a sample collectable from a living organism in a minimally invasive manner; and acquiring information concerning the living organism based on the measured amount of nicotinamide metabolite.

### CROSS REFERENCES TO RELATED APPLICATIONS

The present disclosure claims priority to Japanese Priority Patent Application JP 2010-175710 filed in the Japan Patent Office on Aug. 4, 2010, the entire contents of which is hereby incorporated by reference.

### BACKGROUND

The present disclosure relates to biological information acquisition methods, specifically to methods of acquiring information concerning a living organism based on the amounts of nicotinamide metabolites in a sample collected from the living organism.

Nicotinamide adenine dinucleotide (NAD) is a well known coenzyme of a redox reaction in cellular energy metabolism. NAD+ (nicotinamide adenine dinucleotide, oxidized form) is biosynthesized from nicotinamide (NA) ingested as a nutrient. Specifically, NA is converted into nicotinamide mononucleotide (NMN) by nicotinamide phosphoribosyltransferase (Nampt), and the NMN is converted into NAD+ by nicotinamide mononucleotide adenyltransferase (Nmnat) (see FIG. 14).

In addition to being converted into NADH (nicotinamide adenine dinucleotide, reduced form) in a redox cycle, the NAD+ is converted back into NA by being degraded into NA and O-acetyl-ADP-ribose with enzymes such as sirtuin (SIRT1) (see FIG. 14).

The NAD metabolism, once thought as a phenomenon confined only within the cells, is beginning to be looked at as a process, including transport, that also takes place outside of the cells. The concentrations of NAD+, NADH, and NMN in blood are maintained at high levels on the order of several ten micromoles, an amount ten times or greater than the blood NA concentration, which is only several micromoles. The blood NAD+, NADH, and NMN concentrations do not vary with the intake of NA (see Consideration of diurnal variations in human blood NAD and NADP concentrations, J Nutr Sci Vitaminol (Tokyo), 2009, June; 55(3): 279-81). It is thus believed that the concentrations of NAD+, NADH, and NMN in blood are maintained independent of NA, and that their demand and supply are controlled between cells and tissues, and between tissues and organs. In fact, it is known that the Nampt that converts NA into NMN is hardly expressed in nerve cells or pancreatic β cells, and that these cells, unable to synthesize NAD+ by themselves, rely on the extracellular supply of NAD+ (see Nampt/PBEF/Visfatinregulates insulin secretion in beta cells as a systemic NAD biosynthetic enzyme, Cell Metab, 2007, November; 6(5): 363-75, and Stimulation of nicotinamide adenine dinucleotide biosynthetic pathways delays axonal degeneration after axotomy, J Neurosci, 2006, Aug. 16; 26(33): 8484-91).

Concerning the nicotinamide metabolites (NA, NMN, NAD+, NADH), it is known that the amounts of NAD+ and NADH, or their ratio (NAD+ amount/NADH amount) can be used as an index of cell state and individual health conditions. For example, because the NAD is functional in cellular energy metabolism, cells with small NAD+ amounts are unable to metabolize fat, and tend to accumulate fat. It is thus believed that information concerning, for example, individual susceptibility to overweight, or the risk of developing metabolic syndrome can be acquired based on the amount of NAD+. Further, cancer cells require large amounts of energy, and thus contain large amounts of NADH. Cells with excessively large amounts of NADH can thus be determined as being cancerous. In this connection, an endoscope that can be used to observe a cancer lesion based on the self-fluorescence of NADH is in practical use, and already being used to acquire information concerning the individual risk of developing cancer, or the presence or absence of an onset, based on NADH amount.

The fact that the nicotinamide metabolites are involved in energy metabolism leaves no doubt that these metabolites are associated with fatigue at the cell level. The possible involvement of NAD+ in fatigue at the individual level is also considered, as reported in, for example, Storage and secretion of beta-NAD, ATP and dopamine in NGF-differentiated rat pheochromocytoma PC12 cells, Eur J Neurosci, 2009, September; 30(5): 756-68, Epub 2009 Aug. 27, which describes a mechanism by which the nerve cells accumulate NAD+ in the same manner as for the neurotransmitter, and release NAD+ in response to stimuli. Further, organs are known to undergo changes in metabolic state in response to stress, and because the NAD metabolism is considered to also undergo stress-induced changes, it would be possible to acquire information concerning the stress of an individual based on the amounts of nicotinamide metabolites.

As noted above, the nerve cells and pancreatic β cells rely on the external NAD+ supply. Among different cell types, the nerve cells demand the greatest amount of energy, requiring large amounts of NAD+ for their nerve activities. In NA deficiency (pellagra), dementia is recognized as the most common symptom, and NA deficiency is considered to be a factor that causes abnormalities in the energy metabolism of nerve cells. This suggests the possibility that information concerning the risk of developing dementia, or the presence or absence of an onset might be acquired based on NAD+ amount.

The pancreatic β cells metabolize the intake sugar, and secrete insulin by sensing blood glucose levels. Because the sensing of the sugar requires NAD+, NA is also used as therapeutic drugs for type I diabetes mellitus. Thus, it is considered possible to acquire information concerning the risk of developing diabetes mellitus, or the presence or absence of an onset, based on the amounts of nicotinamide metabolites.

There have been reports that the NMN concentration in the blood of rodents lowers with aging, suggesting the link between the age-related decrease in blood NMN level and dementia or diabetes mellitus (see Age-associated loss of Sirt1-mediated enhancement of glucose-stimulated insulin secretion in beta cell-specific Sirt1-overexpressing (BESTO) mice, Aging Cell, 2008, January; 7(1):78-88, Epub 2007 Nov. 14).

As described above, it is considered possible to acquire useful biological information, such as information concerning the risk of developing diseases such as metabolic syndrome, cancer, dementia, and diabetes mellitus, or the presence or absence of an onset, and information concerning the state of fatigue or stress, based on the amounts of nicotinamide metabolites (NA, NMN, NAD+, NADH).

Known methods of acquiring biological information such as the stress, affectivity, and menstrual cycle of a living organism include biological information acquisition methods that are based on psychological evaluations involving, for example, questioning and sensory questionnaires, physiological tests measuring, for example, brain waves or myoelectricity, and behavior measurements involving the use of, for example, a work record. For example, JP-A-2006-94969 discloses a technique that determines the menstrual cycle based on heart rates. Japanese Patent No. 2582957 discloses a life activity monitoring system that monitors body temperature fluctuations and heart rates.

Simpler techniques are also developed that acquire information concerning a living organism with the use of a physiologically active substance contained in blood, urine, or saliva as an index. For example, JP-A-11-38004 discloses a method for quantifying stress using the concentration of adrenal cortical steroid and/or its metabolic products in saliva as an index. JP-A-2000-131318 discloses a method that allows the stress level to be grasped as either “comfortable” or “uncomfortable” using biological substances such as β-endorphin, dopamine, immunoglobulin A, and prostaglandin D2 contained in blood or the like as an index. The biological information acquisition methods in which the physiologically active substances contained in blood, urine, or saliva are used as an index are advantageous, because these methods are simpler than methods involving psychological evaluations, physiological tests, or behavior measurements, and do not require large devices.

Not far back in history, humans in a given society had more or less the same lifestyle. To be more specific, we used to get in and out of bed at about the same time, eat at about the same times of day, and basically work in the same time of day. Today, people lead lives by freely choosing their own lifestyles. Modes of daily habits, such as wake-up time and bedtime, diet, and eating time vary greatly from one individual to another. Further, more convenience and diversification of work style have created a large group of people greatly lacking exercise time and exercise intensity.

Such diversification of lifestyle has occurred rather quickly in the very short time period of the last several decades. Being unable to physically adapt to such lifestyle changes, many people have physical and mental complaints. These circumstances have created the situation where each individual needs to appropriately manage his/her health, taking into consideration his/her own lifestyle. Further, the diversification of lifestyle has created a need for the development of goods and services, or sales strategy planning, specific to particular lifestyles.

The health management and the development of goods suited to the lifestyles of different individuals require a means by which a diversity of lifestyles can be expressed and evaluated in a standardized fashion. However, while an index that can be used to individually express and evaluate different modes of daily habits such as wake-up time and bedtime, diet, eating time, and exercise time and exercise intensity is available, no index is available that can be used to comprehensively express and evaluate these daily habits as a lifestyle.

### SUMMARY

As described above, there is a need for a means by which a diversity of lifestyles can be expressed and evaluated in a standardized fashion for health management and for the development of goods in a manner suited for the lifestyles of different individuals. Nicotinamide metabolite amount can be used as an index that reflects diseases such as metabolic syndrome, cancer, dementia, and diabetes mellitus, and the physiological state of a living organism concerning fatigue or stress. Thus, if the nicotinamide metabolite amounts that reflect such physiological states could be measured using a sample collectable from a living organism in a minimally invasive manner such as saliva or urine, it would be possible to acquire useful information, including information concerning the risk of developing diseases such as above, or the presence or absence of an onset, information concerning the state of fatigue or stress, and even information concerning lifestyle.

Use of blood as a sample involves blood collection, which is invasive, and mentally or physically demanding to a living organism. This may be perceived as stress, and may cause changes in the living organism's physiological state, and prevent acquisition of accurate biological information. It is therefore preferable that the sample used for the measurement of nicotinamide metabolite amount be collectable from a living organism in a minimally invasive manner, such as from saliva or urine, as noted above.

It is thus desirable to provide a method for acquiring useful biological information, including information concerning the risk of developing a disease such as metabolic syndrome, or the presence or absence of an onset, information concerning the state of fatigue or other conditions, and information concerning lifestyle, based on the amounts of nicotinamide metabolites in a sample collectable from a living organism in a minimally invasive manner.

According to an embodiment of the present disclosure, there is provided a biological information acquisition method that includes measuring the amount of nicotinamide metabolite in one or more samples collectable from a living organism in a minimally invasive manner and selected from oral mucosa epithelial cell, saliva, and epidermal fluid, and acquiring information concerning the living organism based on the measured amount of nicotinamide metabolite.

In the biological information acquisition method, the nicotinamide metabolite may be one or more selected from nicotinamide, nicotinamide mononucleotide, and nicotinamide adenine dinucleotide.

The biological information acquisition method can be used to acquire biological information concerning one or more selected from metabolic syndrome, cancer, fatigue, stress, dementia, diabetes mellitus, biological rhythm, and lifestyle.

Further, according to another embodiment of the present disclosure, there is provided a nicotinamide metabolite measurement method for measuring an nicotinamide metabolite amount in one or more biological samples selected from oral mucosa epithelial cell, saliva, and epidermal fluid, wherein the nicotinamide metabolite amount reflects the physiological state of the living organism.

In the present disclosure, “information concerning a living organism (biological information)” encompasses a wide range of information concerning the physiological state of an individual living organism. Specifically, the biological information is the information concerning, for example, the risk of developing diseases such as metabolic syndrome, cancer, dementia, and diabetes mellitus, which have possible associations with nicotinamide metabolite amounts, or the presence or absence of an onset. The biological information also means information concerning, for example, the state of fatigue, stress, biological rhythm, or lifestyle.

Further, in the present disclosure, “biological rhythm” means the autonomously oscillating periodic rhythm seen in biological phenomena. The biological rhythm governs a wide range of rhythms seen in various biological phenomena, controlling the physiological state of a living organism. For example, the well known circadian rhythm, with a cycle of roughly 24 hours, governs the sleep-wake rhythm, and the diurnal variation rhythms of body temperature, blood pressure, and hormone secretion level, controlling these physiological states. The biological rhythms are controlled by a group of genes called “clock genes”. The clock genes function as an “internal clock”, whereby the behaviors of these genes, including expression, activity, and localization, autonomously vary (oscillate) in a periodic fashion, controlling the biological rhythms, and various physiological states controlled by the biological rhythms.

Further, in the present disclosure, “lifestyle” means the ways one leads a daily life as defined by various modes of daily habits, such as the wake-up time and bedtime for sleeping, the diet and eating time for eating, and the exercise time and exercise intensity for exercising. Note that “daily habit” encompasses a wide range of activities engaged in a daily life, including sleeping, eating, exercising, bathing, drinking, smoking, commuting to school or work, and watching televisions and monitors.

The present disclosure provides the method for acquiring useful biological information, including information concerning the risk of developing a disease such as metabolic syndrome, or the presence or absence of an onset, information concerning the state of fatigue or other conditions, and information concerning lifestyle, based on the amount of nicotinamide metabolite in a sample collectable from a living organism in a minimally invasive manner.

Additional features and advantages are described herein, and will be apparent from the following Detailed Description and the figures.

### BRIEF DESCRIPTION OF THE FIGURES

FIGS. 1A and 1B are diagrams explaining a method of acquiring a physiologically active substance from the skin surface of a finger.

FIG. 2 is a graph representing the results of the measurement of NAD+ and NADH amounts in oral mucosa epithelial cells (Test Example 1).

FIG. 3 is a graph representing the results of the measurement of cortisol amounts in saliva (Test Example 1).

FIG. 4 is a graph representing the results of the measurement of CLOCK/BMAL1 expression level in oral mucosa epithelial cells (Test Example 1).

FIG. 5 is a graph representing the results of the measurement of NAD+ and NADH amounts in saliva and epidermal fluid (Test Example 2).

FIGS. 6A to 6C are chromatograms representing the results of detecting NMN, NAD+, and NA in epidermal fluid (Test Example 2).

FIG. 7 is a graph representing the results of the measurement of nicotinamide metabolite amounts in epidermal fluid after an exercise load (Test Example 3).

FIGS. 8A and 8B are graphs representing the results of the measurement of nicotinamide metabolite amounts in epidermal fluid after waking up (Test Example 3).

FIGS. 9A and 9B are graphs representing the results of the measurement of nicotinamide metabolite amounts in epidermal fluid after eating (Test Example 3).

FIG. 10 is a graph representing the results of the measurement of nicotinamide metabolite amounts in epidermal fluid after the intake of alcohol (Test Example 3).

FIGS. 11A and 11B are graphs representing the correlation between aging and the amounts of nicotinamide metabolites in epidermal fluid (Test Example 3).

FIGS. 12A and 12B are graphs representing the results of the measurement of nicotinamide metabolite amounts on the horny layer of the epidermis surface of a cultured skin after adding cortisol to the culture solution (Test Example 4).

FIGS. 13A and 13B are graphs representing the results of the measurement of nicotinamide metabolite amounts on the horny layer of the epidermis surface of a cultured skin after adding glucose to the culture solution (Test Example 4).

FIG. 14 is a diagram explaining the metabolic pathway of nicotinamide metabolites.

### DETAILED DESCRIPTION

Embodiments of the present application will be described below in detail with reference to the drawings.

The inventor of the present disclosure conducted various studies with the intention to acquire biological information such as information concerning the risk of developing a disease such as metabolic syndrome, or the presence or absence of an onset, information concerning the state of fatigue, and information concerning lifestyle, using nicotinamide metabolite amount as an index. The studies revealed new findings, as follows.

(1) The amount of nicotinamide metabolite in oral mucosa epithelial cells shows changes that reflect the physiological state of a living organism.

(2) Detection of nicotinamide metabolites is also possible from saliva or epidermal fluid.

(3) The amount of nicotinamide metabolite in epidermal fluid shows changes that reflect the lifestyle of a living organism.

Concerning the finding (1), (1a) the inventor of the present disclosure has revealed that the amounts of NAD+ and NADH in oral mucosa epithelial cells show diurnal variations that involve a transient increase immediately after waking up, as with the case of cortisol amount in saliva. (1a) It has also been revealed that the diurnal variations in the amounts of NAD+ and NADH in oral mucosa epithelial cells are antiphase to the diurnal variations in the expression levels of the clock gene CLOCK/BMAL1 conjugates in oral mucosa epithelial cells.

It is well known that the secretion amounts of cortisols such as cortisol, corticosterone, and cortisone into saliva have a correlation with the state of stress of a living organism (see Stimulation of nicotinamide adenine dinucleotide biosynthetic pathways delays axonal degeneration after axotomy, J Neurosci, 2006, Aug. 16; 26(33): 8484-91). CLOCK and BMAL1 are representative clock genes that control the physiological state of a living organism by broadly governing the biological rhythms seen in various biological phenomena such as sleeping and awakening, body temperature, blood pressure, and hormone secretion.

The fact that the NAD+ and NADH amounts in oral mucosa epithelial cells show changes common to those seen in cortisols and CLOCK/BMAL1 suggests that the changes in NAD+ and NADH amounts also reflect the physiological state of a living organism, and can thus be used as an index of the physiological state as in the cortisols and CLOCK/BMAL1.

Concerning the finding (3), the amounts of NAD+ and NADH in epidermal fluid were found to change in a manner that reflects the modes of daily habits, including sleeping, eating, and exercising, and the age of a living organism.

The inventor of the present disclosure, based on these findings, completed a biological information acquisition method that measures the amount of nicotinamide metabolite in oral mucosa epithelial cells or epidermal fluid collectable from a living organism in a minimally invasive manner, and that acquires information concerning the living organism based on the measured amount of nicotinamide metabolite. Further, concerning the finding (2), the inventor found that detection of nicotinamide metabolites was also possible from saliva, and devised a method for acquiring biological information using saliva as a sample collectable from a living organism in a minimally invasive manner.

Concerning the findings (1) and (3), measurements of nicotinamide metabolite amounts in various types of cells have been performed. However, there is no report that suggests that the amount of nicotinamide metabolite in oral mucosa epithelial cells or epidermal fluid collectable from a living organism in an easy, minimally invasive manner shows changes that reflect the physiological state of a living organism.

Further, concerning the finding (2), there is no report of the detection of nicotinamide metabolites from noncellular samples, such as saliva and epidermal fluid, other than blood (blood plasma). The mechanism by which the nicotinamide metabolites are acquired from epidermal fluid may involve a possibility that nicotinamide metabolites are secreted into, for example, sweat or sebum. There is also a possibility that the nicotinamide metabolites in blood pass through the body surface cells to reach the body surface.

The following describes a preferred embodiment of a biological information acquisition method according to the present disclosure. It should be noted that the embodiment described below is merely an exemplary embodiment of the present disclosure, and should not be construed to restrict the scope of the present disclosure. Description is given in the following order.

1. Sample Collection

(1) Collection of oral mucosa epithelial cells

(2) Collection of saliva

(3) Collection of epidermal fluid

2. Measurement of Nicotinamide Metabolite Amounts in Sample

(1) In vitro measurement

(2) In vivo measurement

3. Acquisition of Biological Information

1. Sample Collection

The biological information acquisition method of the embodiment of the present disclosure first measures the amount of nicotinamide metabolite in a sample collectable from a living organism in a minimally invasive manner. The biological information, described later, is then acquired based on the measured amount of nicotinamide metabolite. Specifically, oral mucosa epithelial cells, saliva, or body surface fluid are used as a sample collectable from a living organism in a minimally invasive manner. The method of collecting each sample is as follows.

(1) Collection of Oral Mucosa Epithelial Cells

The oral mucosa epithelial cells are an example of a cellular sample that can be collected from a living organism in the least invasive manner. The oral mucosa epithelial cells can be collected by, for example, scraping the cells from the oral mucosa surface using a tool such as a brush and a spatel. The site of collection is preferably the mucosa on the back of the cheek.

The oral mucosa epithelial cells collected with a brush or the like can be collected in a sample tube by washing the brush or the like in a buffer such as a phosphate buffer (PBS) filling the sample tube. Here, the cells are either lysed using, for example, a buffer that contains a surfactant, or physically disrupted and lysed by, for example, sonication in a buffer suspension, so as to prepare a sample solution containing nicotinamide metabolites derived from the oral mucosa epithelial cells.

(2) Collection of Saliva

Saliva is an acellular sample, and is collectable from a living organism in a minimally invasive manner. Saliva can be collected by, for example, placing a filter paper or a capillary tube in the oral cavity, and allowing the saliva to be absorbed into the filter paper or enter the capillary tube. The collected saliva is prepared into a nicotinamide metabolite-containing sample solution either directly or in a buffer.

(3) Collection of Epidermal Fluid

Epidermal fluids such as sweat and sebum are also acellular samples, and are collectable from a living organism in a minimally invasive manner. The epidermal fluid can be collected by contacting a filter paper or a capillary tube to the body surface, and allowing the epidermal fluid to be absorbed into the filter paper or enter the capillary tube. The collected epidermal fluid is prepared into a nicotinamide metabolite-containing sample solution either directly or in a buffer.

Alternatively, a solvent may be contacted to the body surface to collect the epidermal fluid in the solvent and prepare a sample solution. The solvent may be water or various organic solvents, for example, such as ethanol water. The body surface contacted to the solvent is not particularly limited, and the skin surface of, for example, a finger or a palm can be conveniently used.

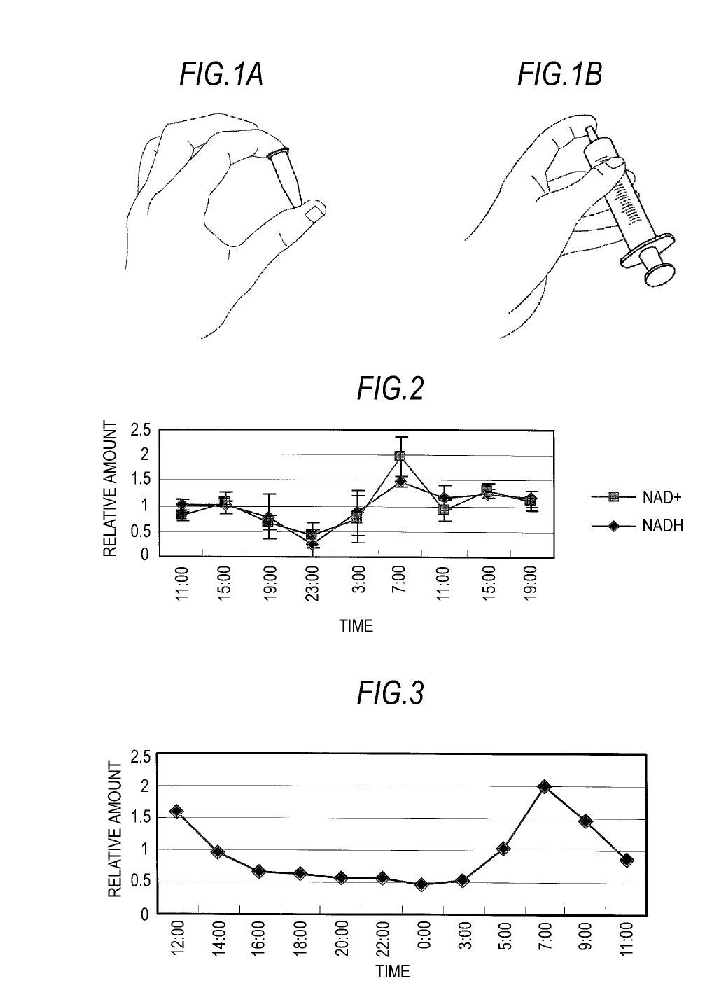

As a specific example of the preferred procedure of acquiring the epidermal fluid, the following describes a method of acquiring epidermal fluid from the skin surface of a finger, with reference to FIGS. 1A and 1B.

FIG. 1A is a diagram illustrating the procedure of acquiring epidermal fluid from the skin surface of index finger using a microtube.

The bottom end of a microtube containing a solvent such as ethanol water is held with the thumb, with the upper opening of the microtube in contact with the tip of the index finger. With the microtube held with the index finger and the thumb, the microtube is inverted to contact the solvent to the skin surface of the index finger. In this way, the epidermal fluid present on the skin surface of the index finger can be collected in the solvent contained in the microtube.

FIG. 1B is a diagram illustrating the procedure of acquiring epidermal fluid from the skin surface of index finger using a syringe.

A syringe charged with a solvent such as ethanol water at the tip is held with the thumb and the middle finger, with the syringe in contact with the tip of the index finger. The piston in the syringe is then pulled with the right hand to create a negative pressure in the syringe. As a result, the skin surface is sucked by the syringe, and the solvent contacts the skin surface of the index finger. In this way, the solvent that has contacted the skin surface can be collected at higher yield based on the negative pressure of the syringe than in the method using a microtube illustrated in FIG. 1A.

In the collection procedures of FIGS. 1A and 1B, the epidermal fluid is collected in the solvent directly in contact with the body surface; however, the epidermal fluid may also be collected in the solvent in the following manner. Specifically, for example, a plastic plate is pressed against the body surface in a manner allowing the epidermal fluid on the body surface to attach to the surface of the plastic plate. Then, a solvent is dropped onto the surface of the plastic plate to dissolve and collect the attached epidermal fluid in the solvent.

2. Measurement of Nicotinamide Metabolite Amounts in Sample

The amount of nicotinamide metabolite in the prepared sample solution can be measured as follows.

(1) In Vitro Measurement

The nicotinamide metabolites in a sample solution can be measured by detecting the photoabsorption, fluorescence, or redox potential of the molecules separated by using techniques such as liquid chromatography (HPLC) and capillary electrophoresis. In this method, measurement may be made using the epidermal fluid collected by directly contacting the HPLC mobile phase solvent or capillary electrophoresis gel to the skin surface.

The nicotinamide metabolite amounts also can be measured by detecting the redox potential of NAD+ and NADH in a mixture of a sample solution and a reaction liquid that contains a redox enzyme and a substrate plus the coenzyme NAD+ and NADH. For example, diaphorase and 2-amino-1,4-naphthoquinone (ANQ) may be used as the redox enzyme and the substrate. The measurement of nicotinamide metabolite amounts using the redox potential of NAD+ and NADH is performed after the NMN, converted from NA in a sample solution with Nampt, is converted into NAD+ using Nmnat. In this method, measurement may be made after the oral mucosa epithelial cells collected with a brush or the like, or the collected saliva are directly collected in the reaction liquid. Further, measurement may be made using the epidermal fluid collected by directly contacting the reaction liquid to the skin surface.

Further, the measurement of nicotinamide metabolite amount may be made using a colorimetric analysis that utilizes the enzyme cycling reaction. Measurement using colorimetric analysis may be performed with a commercially available kit (see Experiment Example 1 described later).

(2) In Vivo Measurement

In the embodiment of the present disclosure, the amounts of nicotinamide metabolites are measured using the oral mucosa epithelial cells, saliva, and epidermal fluid collected from a living organism. However, concerning the oral mucosa epithelial cells, the measurement method is not limited to the measurement of the nicotinamide metabolite amounts in the collected and separated cells, and the amounts of nicotinamide metabolites in the oral mucosa epithelial cells may be measured in situ, without separating the cells from the living organism.

An example of the method that measures the nicotinamide metabolite amounts in the oral mucosa epithelial cells in situ is the method that performs the measurement based on the self-fluorescence of NADH. NADH exhibits self-fluorescence at an excitation wavelength of about 350 nm, and an emission wavelength of about 450 nm. Quantification of NADH based on fluorescence intensity is thus possible by detecting the self-fluorescence of the NADH excited in the epithelial cells by multiphoton excitation following the irradiation of the oral mucosa with a femtosecond laser. The use of multiphoton excitation enables the measurement of nicotinamide metabolite amount in the cells without injuring the mucosa. Note that this method is also applicable to the in situ measurement of nicotinamide metabolite amounts in the skin cells.

For the in situ measurement of nicotinamide metabolite amounts in epidermal fluid, a method can be used in which, for example, an electrode or an FET sensor immobilizing an enzyme that specifically reacts (interacts) with the nicotinamide metabolites is contacted to the skin surface.

3. Acquisition of Biological Information

The measured amount of nicotinamide metabolite can be used as an index that reflects the physiological state of a living organism. The biological information acquisition method of the embodiment of the present disclosure can be used to acquire useful biological information such as information concerning the risk of developing diseases such as metabolic syndrome, cancer, dementia, and diabetes mellitus, which have possible associations with nicotinamide metabolite amounts, or the presence or absence of an onset, information concerning the state of fatigue or stress, and information concerning lifestyle, based on the nicotinamide metabolite amount.

The biological information acquisition method uses oral mucosa epithelial cells, saliva, or body surface fluid as a sample collectable from a living organism in a minimally invasive manner, and can thus be used to conveniently acquire biological information. When body surface fluid is used as a sample, information that reflects the physiological state of a subject can be acquired with particular accuracy, without making the subject too worry of the collection procedure. The biological information acquisition method of the embodiment according to the present disclosure can thus be used to find the health conditions of a living organism for the diagnosis, prevention, and prognosis of diseases, fatigue, and stress. Further, the method can be used to provide an index that reflects modes of daily habits such as wake-up time and bedtime, diet, eating time, and exercise time and exercise intensity, and can thus provide means by which a diversity of lifestyles can be expressed and evaluated in a standardized fashion.

### EXAMPLES

### Test Example 1

1. Measurement of NAD+ and NADH Amounts in Oral Mucosa Epithelial Cells

The epithelial cells were collected from the oral mucosa of a single subject (adult male), using a brush (CytoSoft cytology brush; Medical Packaging Corp.). The cells were collected for a total of nine times at 4-hour intervals. The first collection was made at 7:00 immediately after the subject woke up, followed by a series of collections at 11:00, 15:00, and 19:00, and at 23:00 just before bedtime, and finally at 3:00 after temporarily waking up the subject. Three samples were collected each time.

The cells collected at each collection time were disrupted by being lysed in a buffer (20 mM Tris-HCl, pH 7.5, 150 mM NaCl, 1 mM EDTA, and 1% sucrose monolaurate). The NAD+ and NADH amounts were then measured using 2 μl of the resulting cell lysate with a commercially available colorimetric analysis kit (Amplite Colorimetric NAD/NADH Assay Kit; ABD Bioquest Inc.). The measurement results are presented in FIG. 2.

2. Measurement of Cortisol Amount in Saliva

Saliva was collected using a Sorbette (Salimetrics). After soaking the Sorbette cotton with saliva, the plastic handle portion was cut in half, and set in a 2.0-ml tube, which was then centrifuged to collect the saliva. The amount of cortisol was measured using the saliva with a commercially available enzyme immunoassay (EIA) kit (Salivary Cortisol EIA Kit; Salimetrics). The measurement results are presented in FIG. 3.

3. Measurement of CLOCK/BMAL1 Expression Levels in Oral Mucosa Epithelial Cells

The expression levels of CLOCK/BMAL1 conjugates were measured according to the method of JP-A-2008-67694, using a part of the cell lysate obtained by disrupting the collected oral mucosa epithelial cells.

First, an oligoDNA with a fluorescent dye (FITC) and a quencher substance (BHQ) attached to the 5′-end and 3′-end, respectively, of the DNA binding sequence (SEQ ID NO: 1) of the CLOCK/BMAL1 conjugates was synthesized as a detection nucleic acid chain (see Table 1). The base sequence of SEQ ID NO: 1 is the recognition sequence of the CLOCK/BMAL1 conjugates as a transcription factor. The CLOCK/BMAL1 conjugates recognize this sequence, and exhibit transcriptional activity upon binding to the DNA. An oligoDNA with the base sequence of SEQ ID NO: 2 was also synthesized as the complementary nucleic acid chain that binds to the detection nucleic acid chain to form a double strand (see Table 1).

TABLE 1

| | | |
|---|---|---|
| Detection | 5′ FITC-acccag(AP) | SEQ ID NO: 1 |
| nucleic | ccacgtgc-BHQ 3′ | |
| acid chain | | |
| Complementary | 5′ gcacgtggatgggt 3′ | SEQ ID NO: 2 |
| nucleic acid | | |
| chain | | |

The sixth base from the 5′-end of the detection nucleic acid chain represents the “AP site (Apurinic/Apyrimidinic site)” lacking a guanine. The AP site is the site of the nucleic acid chain lacking a base (dropping of a base). The AP site is specifically cut by AP-endonuclease, which recognizes the double-strand, and nicks (cuts) the AP site.

Then, anti-BMAL1 antibodies (Santa Cruz Biotechnology) were immobilized on magnetic beads (micromer-M [PEG-COOH]; mcromod Partikel technologie) using a PolyLink-Protein Coupling Kit for COOH Microparticles (Polyscience) to prepare antibody magnetic beads.

The cell lysate was centrifuged (16,000 g, 10 min) to precipitate the insoluble components. After separating the supernatant, the protein concentration of each sample was made equal by measuring absorbance (280 nm).

The samples were diluted with PBS (pH 7.5) containing an equal amount of protease inhibitor cocktail, and with 0.05% Tween 20 (PBS-T), and incubated at 4° C. for 2 hours with addition of the antibody magnetic beads, so as to bind the CLOCK/BMAL1 conjugates to the antibody magnetic beads. The antibody magnetic beads were collected, and the molecules that had non-specifically bound to the beads were washed with PBS-T.

The antibody magnetic beads were then suspended in a solution (10 mM Tris-Cl (pH 7.5), 50 mM KCl, 2.5% glycerol, 10 mM EDTA, 0.05% NP-40, 0.05 mg/mL salmon sperm DNA) containing the detection nucleic acid chain and the complementary nucleic acid chain (0.25 μM each) forming a double strand, and incubated at 37° C. for 1 hour to bind the detection nucleic acid chain to the CLOCK/BMAL1 conjugates captured on the beads. The antibody magnetic beads were collected, and the molecules that had non-specifically bound to the CLOCK/BMAL1 conjugates were washed with PBS-T.

The antibody magnetic beads were then suspended in water, and incubated at 80° C. to dissociate the double strand of the detection nucleic acid chain and the complementary nucleic acid chain bound to the CLOCK/BMAL1 conjugates.

The dissociated double strand of the detection nucleic acid chain and the complementary nucleic acid chain was suspended in 20 mM Tris-Acetat, 10 mM Mg-Acetat, 50 mM KCl, and 1 mM DTT (pH 7.9), and incubated at 37° C. with addition of a single-stranded detection nucleic acid chain (0.2 μM) and AP endonuclease.

During the incubation, the AP endonuclease cuts the AP site of the detection nucleic acid chain, separating the fluorescent dye and the quencher substance and causing the free fluorescent dye to fluoresce. At the same time, the complementary nucleic acid chain that had bound to the cut detection nucleic acid chain becomes a single strand, and binds to another single-stranded detection nucleic acid chain to form a new double strand. The AP endonuclease cuts the newly formed double strand and frees the fluorescent dye. The fluorescence intensity increases over time as this procedure is repeated. The time-dependent increase in fluorescence intensity has a positive correlation with the amount of CLOCK/BMAL1 conjugate present in the cell lysate.

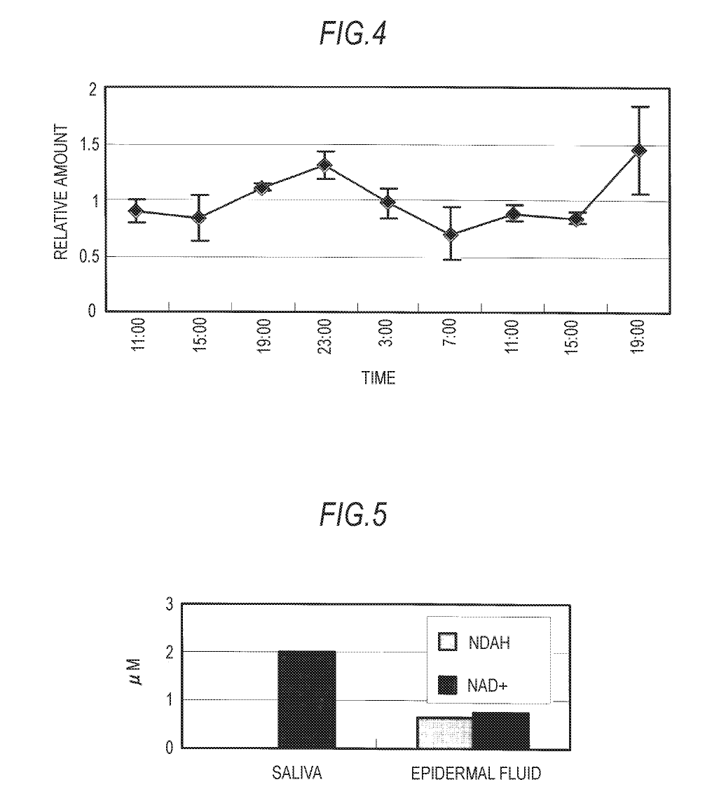

FIG. 4 represents the result of the measurement of the time-dependent fluorescence intensity increase during the incubation.

4. Discussion

The NAD+ and NADH amounts in the oral mucosa epithelial cells in FIG. 2 show a transient increase at 7:00 on rising. This is a characteristic change that coincides with the change in the amount of cortisol in saliva represented in FIG. 3. The cortisol levels in saliva and blood are known to transiently increase under stress, and also on rising to the levels equal to or even greater than the levels under stress.

Further, NAD+ and NADH amounts in the oral mucosa epithelial cells in FIG. 2 fluctuate according to the circadian rhythm, reaching the maximum at around 11:00 and falling to the minimum at around 23:00, setting aside the transient increase on rising. This is antiphase to the circadian rhythm followed by the changes in the amount of the CLOCK/BMAL1 conjugate in the oral mucosa epithelial cells represented in FIG. 4.

### Test Example 2

1. Measurement of NAD+ and NADH Amounts in Saliva and Epidermal Fluid

The amounts of NAD+ and NADH in saliva were measured using a portion of collected saliva. The amounts of NAD+ and NADH in epidermal fluid were also measured using collected epidermal fluid. The epidermal fluid was collected according to the following procedure.

The tip of index finger was gently wiped with a paper towel soaked with pure water. The bottom end of a microtube containing 50 μL of pure water was held with the thumb, with the upper opening of the microtube in contact with the tip of the index finger (see FIG. 1A). With the microtube held with the index finger and the thumb, the microtube was inverted to contact the pure water to the skin surface of the index finger for 1 minute.

FIG. 5 represents the results of measurements of the NAD+ and NADH amounts in the collected saliva and epidermal fluid using the colorimetric analysis kit. NAD+ and/or NADH were detected in both saliva and epidermal fluid.

2. Detection of NMN, NAD+ , and NA Amounts in Epidermal Fluid

Epidermal fluid was used for the detection of NMN, NAD+, and NA by liquid chromatography.

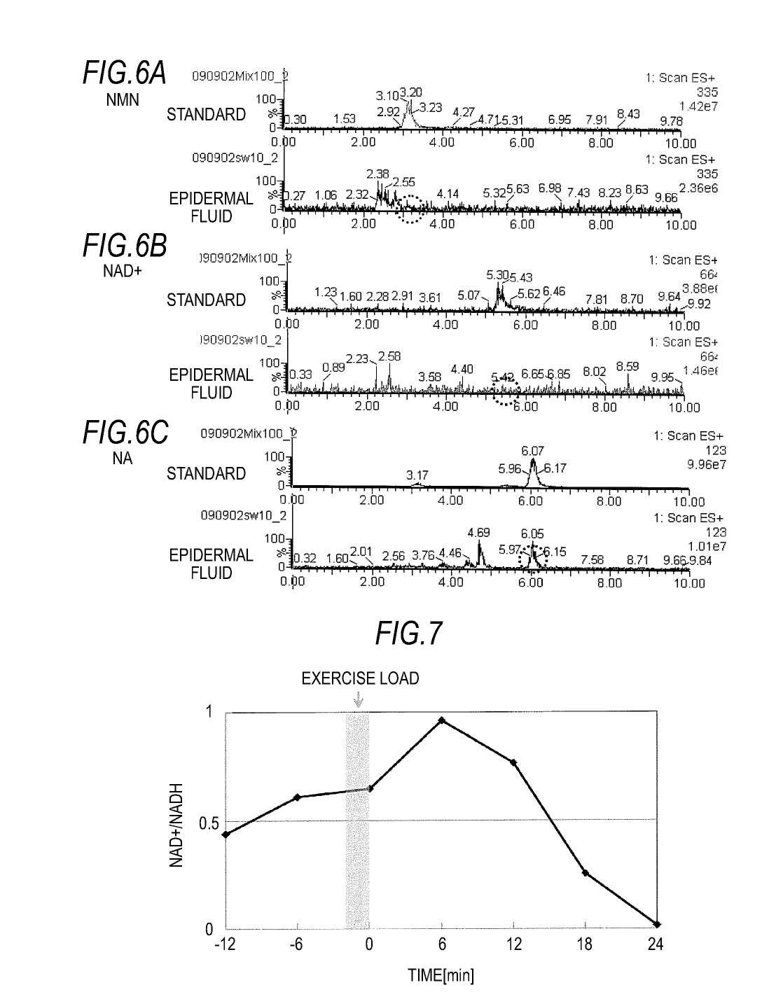

NMN, NAD+, and NA were detected by mass analysis after separation by liquid chromatography (UPLC/MS; Waters), using reverse-phase C18 column (Acquity UPLC BEH; Waters), mobile phase 5 mM ammonium acetate, 0.25% acetic acid, and 1% methanol. The results are presented in FIGS. 6A to 6C.

In FIG. 6A, the same peak observed in the standard NMN was confirmed in the MS spectrum for the column retention time corresponding to the molecular weight 335 of NMN (circled in the figure). In FIGS. 6B and 6C, the same peaks observed in the standard NAD+ and NA were confirmed in the MS spectra for the column retention times corresponding to the molecular weights 664 and 123 of NAD+ and NA, respectively (circled in the figure).

### Test Example 3

1. Correlation between Nicotinamide Metabolite Amount in Epidermal Fluid and Exercise

A single subject was put under an exercise load for 2 minutes with an ergometer, and the epidermal fluid was collected at 6-min intervals. The amounts of NAD+ and NADH in the epidermal fluid were then measured. The collection of epidermal fluid and the measurement of NAD+ and NADH amounts were performed according to the methods described in Test Example 2.

The results are presented in FIG. 7. The NAD+/NADH ratio shows a transient increase immediately after the exercise load. The results thus confirmed that the amounts of nicotinamide metabolites in the epidermal fluid could change in a manner that reflects the exercise time or exercise intensity of a living organism.

2. Correlation between Nicotinamide Metabolite Amounts in Epidermal Fluid and Sleep

Epidermal fluid was collected from a single subject at 7:00 immediately after waking up, and at 7:45, 8:30, 9:30, and 10:30. The NAD+ and NADH amounts in the epidermal fluid were then measured. The collection of epidermal fluid and the measurement of NAD+ and NADH amounts were performed according the methods described in Test Example 2.

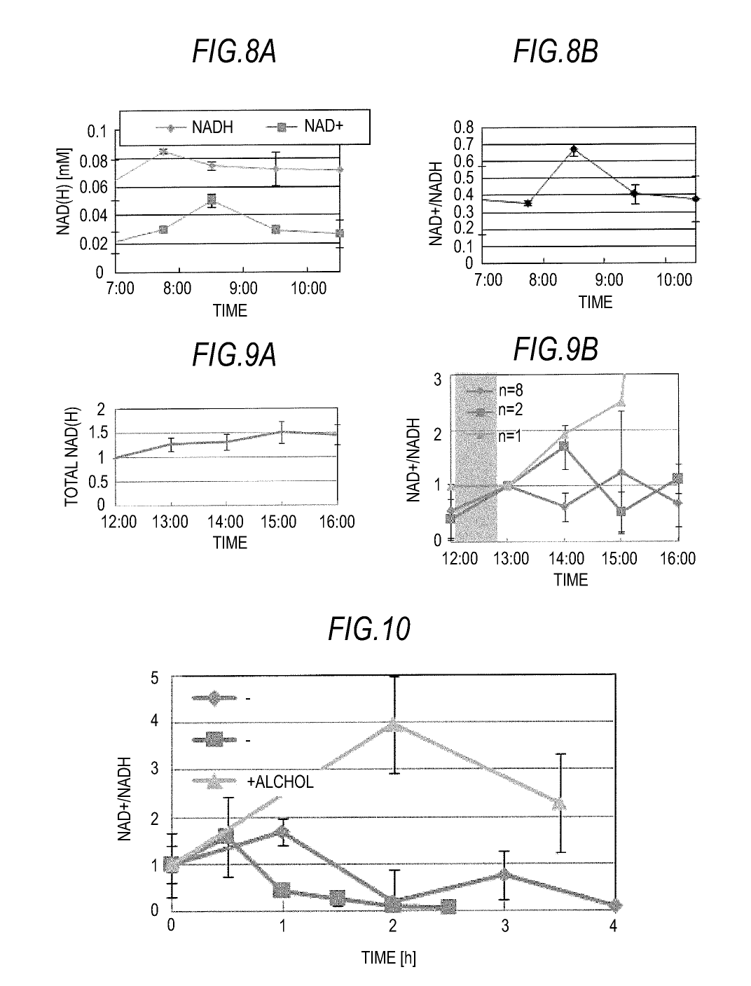

The results are presented in FIGS. 8A and 8B. The NAD+ and NADH amounts (see FIG. 8A), and the NAD+/NADH ratio (see FIG. 8B) both show a transient increase on rising. Such a transient increase on rising is also seen in blood cortisol concentration by a phenomenon known as CAR (Cortisol Awakening Response). There is a report that CAR can be used as an index of sleep quality (effect) (see J. Psychosom. Res., 2000, Vol. 49, No. 5, pp. 335-42, Psychoneuroendocrinology, 2009, Vol. 34, No. 10, p. 1476-85, Biol. Psychol., 2009, Vol. 82, No. 2, pp. 149-55, J. Endocrinol. Invest., 2008, Vol. 31, No. 1, pp. 16-24). It was therefore confirmed that the nicotinamide metabolite amounts in epidermal fluid change by reflecting the wake-up time of a living organism, and can thus be used as an index of sleep quality evaluation.

3. Correlation between Nicotinamide Metabolite Amounts in Epidermal Fluid and Eating

Eleven subjects were allowed to eat the same food during the time period of from 12:00 to 13:00, and the epidermal fluid was collected at 12:00, 13:00, 14:00, 15:00, and 16:00. FIGS. 9A and 9B represent the results of the NAD+ and NADH amount measurements performed according to the method described in Test Example 2.

As shown in FIG. 9A, the sum of NAD+ and NADH amounts (total NAD (H)) increased, with the peak appearing at 15:00, two hours after eating. Decreases in NAD+/NADH ratio were observed in almost all subjects (n=10/11) at hour 1 (n=8) and hour 2 (n=2) post-eating (see FIG. 9B). The transient increase in NAD+/NADH ratio was more enhanced with the intake of alcohol while eating than when no alcohol was taken (see FIG. 10). These results confirmed that the amounts of nicotinamide metabolites in the epidermal fluid change by reflecting the eating time or diet of a living organism.

4. Correlation between Nicotinamide Metabolite Amounts in Epidermal Fluid and Aging

The daily average of the NAD+ and NADH amounts was calculated for each of the eleven subjects of the foregoing test in which the epidermal fluid was collected from the subjects who had the same food from 12:00 to 13:00.

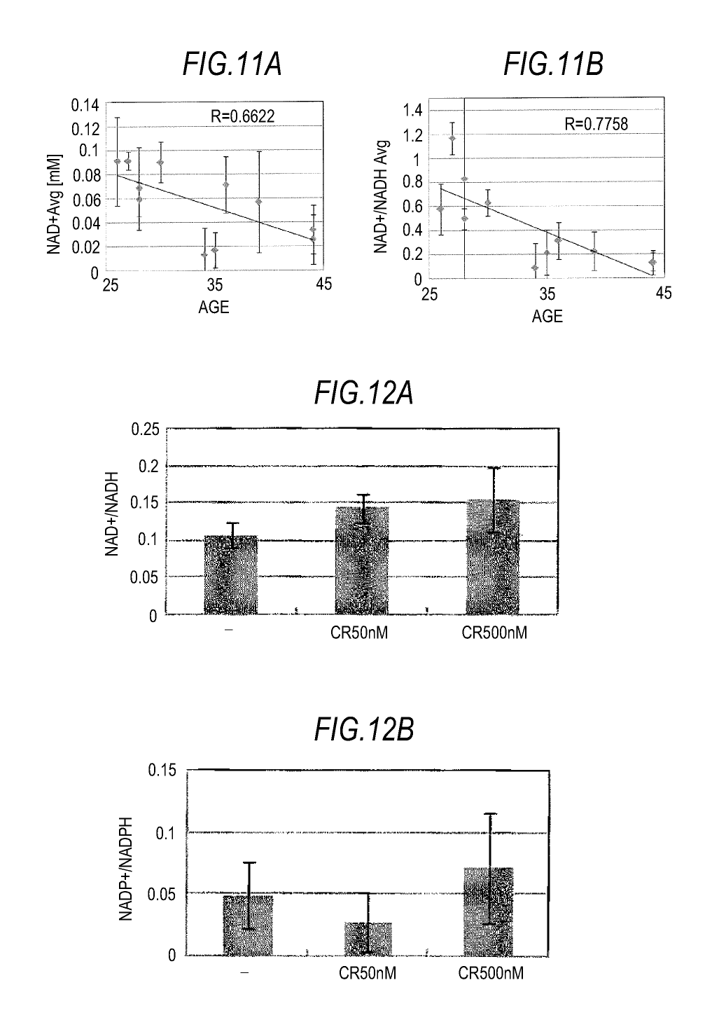

The results are shown in FIGS. 11A and 11B. The NAD+ amount (FIG. 11A) and the NAD+/NADH ratio (FIG. 11B) in epidermal fluid were found to decrease with aging.

### Test Example 4

1. Acquisition of Nicotinamide Metabolites from Cultured Skin Surface

Assessment was made as to the ability of a cultured skin to secrete nicotinamide metabolites on the horny layer of epidermis surface in response to cortisol or glucose.

A three-dimensional culture skin (Kurabo; EPI-200X) was cultured with its dermis side in contact with the culture solution. Glucocorticoid (cortisol) was added to the culture solution to make the final concentration 50 nM or 500 nM. After 1 hour, 150 μL of PBS was contacted to the epidermis side, and the sample was collected. The amounts of NAD+, NADH, NADP+, and NADPH in the sample were then measured using commercially available colorimetric analysis kits (Amplite Colorimetric NAD/NADH Assay Kit, Amplite Colorimetric NADP/NADPH Assay Kit; ABD Bioquest Inc.).

The results are shown in FIGS. 12A and 12B. The NAD+/NADH ratio (FIG. 12A) increased concentration dependently with the glucocorticoid added to the culture solution, whereas there was no change in the NADP+/NADPH ratio (FIG. 12B).

Then, after adding low-concentration (final concentration of 1,500 mg/L) or high-concentration (4,500 mg/L) glucose to the culture solution, 150 μL of PBS was contacted to the epidermis side, and the NAD+, NADH, NADP+, NADPH amounts in the collected sample were measured.

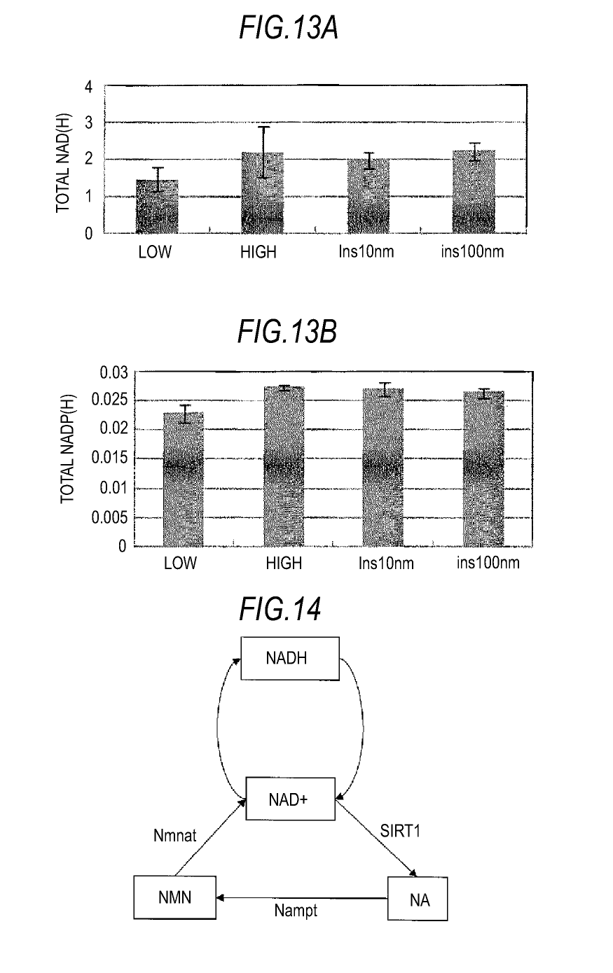

The results are presented in FIGS. 13A and 13B. The total NAD (H) amount (the sum of NAD+ amount and NADH amount) increased more with the addition of the high-concentration glucose (“High” in the figure) than with the low-concentration glucose (“Low”), regardless of whether insulin was added or not (FIG. 13A). The total NADP (H) amount (the sum of NADP+ amount and NADPH amount) also increased in the same manner (FIG. 13B).

It is known that stress or the intake of alcohol increases the blood cortisol concentration. There is also a report that the transient increase in blood cortisol concentration on rising (CAR) can be used as an index of sleep quality (effect) evaluation. The results of the present test examples suggest the possibility that the stress- or alcohol-induced increase in blood cortisol concentration, or CAR can be detected as an increase in the nicotinamide metabolite amount measurable on skin surface. It is also considered possible that the increase in blood glucose level after eating also can be detected as an increase in the nicotinamide metabolite amount measurable on skin surface.

The biological information acquisition method of the embodiment according to the present disclosure can be used to acquire biological information, including information concerning the risk of developing diseases such as metabolic syndrome, cancer, dementia, and diabetes mellitus, or the presence or absence of an onset, and information concerning the state of fatigue or stress, and can thus be used for, for example, the diagnosis, prevention, or prognosis of diseases such as above. The method also provides means by which a diversity of lifestyles can be expressed and evaluated in a standardized fashion based on an index that reflects modes of daily habits, such as wake-up time and bedtime, diet, eating time, and exercise time and exercise intensity.

It should be understood that various changes and modifications to the presently preferred embodiments described herein will be apparent to those skilled in the art. Such changes and modifications can be made without departing from the spirit and scope and without diminishing its intended advantages. It is therefore intended that such changes and modifications be covered by the appended claims.

1. A biological information acquisition method comprising:
measuring an amount of nicotinamide metabolite in a sample collectable from a living organism in a minimally invasive manner; and
acquiring information concerning the living organism based on the measured amount of nicotinamide metabolite.

2. The method according to claim 1, wherein the sample is one or more selected from the group consisting of oral mucosa epithelial cell, saliva, and epidermal fluid.

3. The method according to claim 2, wherein the nicotinamide metabolite is one or more selected from the group consisting of nicotinamide, nicotinamide mononucleotide, and nicotinamide adenine dinucleotide.

4. The method according to claim 3, wherein the biological information acquired is information concerning one or more selected from the group consisting of metabolic syndrome, cancer, fatigue, stress, dementia, diabetes mellitus, biological rhythm, and lifestyle.

5. A nicotinamide metabolite measurement method comprising:
measuring an amount of nicotinamide metabolite in one or more biological samples selected from the group consisting of oral mucosa epithelial cell, saliva, and epidermal fluid, wherein the nicotinamide metabolite amount reflects a physiological state of the living organism.
